(19) United States
(12) Patent Application Publication
(10) Pub. No.: US 2008/0118423 A1
(43) Pub. Date: May 22, 2008

(54) Closed loop control of air/fuel ratio in a reformer for modulating diesel exhaust

(76) Inventors: Gerald T. Fattic, Fishers, IN (US); Da Yu Wang, Troy, MI (US)

Correspondence Address: DELPHI TECHNOLOGIES, INC., M/C 480-410-202, PO BOX 5052, TROY, MI, 48007, US

(21) Appl. No.: 11/601,204

(22) Filed: Nov. 17, 2006

(51) Int. Cl. B01D 53/56; A61L 9/00

(52) U.S. Cl. 423/235; 422/30; 422/168

(57) ABSTRACT

A reformer system comprising a hydrocarbon reformer; a fuel supply system; an air supply system; a hydrogen sensor disposed in a reformate exhaust stream from the reformer; and a reformer controller for receiving input from the hydrogen sensor and setting the flow values for fuel and air to provide a desired O/C ratio in the reformate stream. A protocol of varying fueling rates is run in which a calibration relating hydrogen sensor values to O/C ratio is generated and is programmed into the controller. From this calibration, a fueling rate is selected which provides an O/C ratio within a predetermined range. The reformer system is especially useful for regeneration of a nitrogen oxides trap in a diesel exhaust system. The calibration protocol may be run during engine operation and can adjust the fueling rate when different diesel fuel mixtures are presented.

### TECHNICAL FIELD

The present invention relates to reformers for catalytically converting hydrocarbons into hydrogen-containing reformate; more particularly, to methods and apparatus for controlling the ratio of air to fuel during various phases of reformer operation; and most particularly, to a method and apparatus for controlling the air/fuel ratio by measuring the mole fraction of hydrogen in the reformate and feeding back such measurement to a fuel and air supply controller in a closed-loop mode.

### BACKGROUND OF THE INVENTION

Catalytic reformers for converting hydrocarbons (referred to herein as “fuel”) and air to reformate are well known, air being a ready source of oxygen for the reforming process in exothermic mode. Such reformate typically comprises hydrogen, carbon monoxide, nitrogen, and residual hydrocarbons. The flow rates of fuel and air typically are monitored and controlled by electronic control means, such as a programmable controller or a computer.

In the known art, fuel flow rate is provided in open-loop control based upon the measured mass air flow rate at the inlet to the system and a resultant base pulse width of a fuel injector. There is no feedback control derived from the degree of accuracy of the resultant air-to-fuel (A/F) ratio. The actual A/F ratio delivered to the reformer catalyst is not known but rather is inferred from the measured inlet air mass flow rate and the expected fuel mass flow rate from the fuel injector. Because of variations in production hardware, the air and fuel control setpoints can have associated errors that can result in poor combustion and excess fuel deposition on the interior walls of the reformer, especially during a start-up combustion phase.

In the automotive prior art, a diesel engine is typically provided with a trap in the exhaust flow stream for adsorbing oxides of nitrogen (referred to herein as an NOx trap) that are generated during normal engine combustion. A shortcoming of prior art NOx traps is that, while they are relatively efficient collectors of NOx, they have relatively little capacity before becoming saturated and inoperative, requiring regeneration of the adsorbent medium. Such regeneration may be accomplished by passing a reducing atmosphere through the NOx trap to reduce the nitrogen oxides to gaseous nitrogen. Reformate being rich in hydrogen and carbon monoxide represents an excellent regenerative medium, and thus it is known to provide a diesel engine with a catalytic reformer for bleeding reformate into the engine exhaust stream ahead of the NOx trap.

In such a use of a reformer, it is important that the A/F ratio of fuel mixture entering the reformer be controlled such that, on the one hand, no carbon soot is formed (oxygen/carbon (O/C) ratio too low), and on the other hand, the fraction of hydrogen is not significantly reduced (oxygen/carbon ratio too high).

What is needed in the art is an improved means for maintaining at a desired value the ratio of oxygen to carbon in the reformate exhaust of a catalytic hydrocarbon reformer.

What is further needed is a means for automatically adjusting the response of such improved means to compensate for decay in reformer output and change in fuel composition and additives.

It is a principal object of the present invention to provide reformate having a predetermined O/C ratio for injection into a diesel engine exhaust for regeneration of an NOx trap.

### SUMMARY OF THE INVENTION

Briefly described, a reformer system in accordance with the invention comprises a conventional hydrocarbon reformer; a controllable fuel supply system for supplying fuel to the reformer; a controllable air supply system for supplying air to the reformer; a hydrogen sensor disposed in a reformate exhaust stream from the reformer; and a reformer controller for receiving input from the hydrogen sensor and setting the flow values for fuel and air to provide a desired O/C ratio. When the reformer is in warmed-up, steady state mode, a protocol of varying fueling rates is run in which a calibration curve relating hydrogen sensor values to O/C ratio is generated and is programmed into the controller. From this calibration, a fueling rate is selected which provides an O/C ratio within a predetermined range between about 1.05 and 1.10. Continued monitoring of the hydrogen sensor during operation provides continuous feedback control to assure that the O/C ratio remains within the desired range. The reformer system is especially useful in generating reformate for regeneration of a nitrogen oxides trap in a diesel engine exhaust system. The calibration protocol may be run at any time during engine operation and can automatically adjust the fueling rate when different diesel fuel mixtures are presented, for example, those having various additives such as toluene and the like.

### BRIEF DESCRIPTION OF THE DRAWINGS

The present invention will now be described, by way of example, with reference to the accompanying drawings, in which:

FIG. 1 is a schematic drawing showing generation of reformate and entry of reformate into a diesel exhaust stream ahead of an NOx trap;

FIG. 2 is a curve showing predicted production of hydrogen in the reformer shown in FIG. 1 as a function of O/C ratio;

FIG. 3 is a curve showing hydrogen sensor voltage as a function of O/C ratio;

FIGS. 4a and 4b show an auto-calibrate algorithm for reformer warm up;

FIG. 5 is a time-line for auto-calibrate mode for the reformer;

FIG. 6 shows O/C ratio and hydrogen sensor values as a function of fueling rate during auto-calibrate mode;

FIG. 7 is like FIG. 6 but shows the last O/C sensor reading during each read interval;

FIG. 8 is like FIG. 7 but includes the three passes during the auto-calibrate routine during the warm-up period;

FIG. 9 shows the auto-calibrate mode for pulsed reformer operation;

FIG. 10 shows the maximum sensor reading during pulsed operation;

FIG. 11 is an algorithm for auto-calibrate during pulsed operation;

FIG. 12 is an algorithm for pulsed operation after auto-calibrate mode;

FIG. 13 shows recalibration in pulsed operation;

FIG. 14 is two curves of sensor voltage as a function of O/C ratio, showing the effect of aging of the catalyst on hydrogen production in reformate; and

FIG. 15 is two curves showing sensitivity of maximum sensor reading to additives in the diesel fuel.

Corresponding reference characters indicate corresponding parts throughout the several views. The exemplifications set out herein illustrates two preferred embodiment of the invention, and such exemplifications are not to be construed as limiting the scope of the invention in any manner.

### DESCRIPTION OF THE PREFERRED EMBODIMENTS

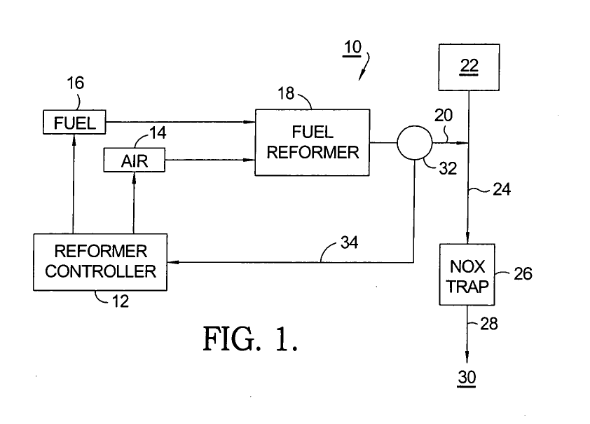

Referring to FIG. 1, an O/C control system 10 in accordance with the invention includes a reformer controller 12 that regulates flows of air 14 and fuel 16 into a hydrocarbon catalytic reformer 18 to produce a reformate 20 containing hydrogen (H₂) and carbon monoxide (CO) as the main species in the output gases, and lower concentrations of carbon dioxide (CO₂), water (H₂O), and methane (CH₄). A diesel engine 22 produces an engine exhaust 24 that may contain oxides of nitrogen. Exhaust 24 passes through NOx trap 26, and exhaust 28 stripped of NOx passes to atmosphere 30. As described below, reformate 20 is added to engine exhaust 24 on a predetermined schedule to reduce NOx trapped by trap 26, thereby regenerating the trapping capability of trap 26. NOx is reduced by reformate to gaseous N₂ which is then swept out of trap 26 by the flow of exhaust 24,28.

A hydrogen sensor 32 is disposed in the flow path of reformate 20 for sensing hydrogen mole percent in the reformate and sending a proportional signal 34 to controller 12 for closed-loop control of flows of air 14 and fuel 16 into reformer 18 responsive to one or more algorithms programmed into controller 12.

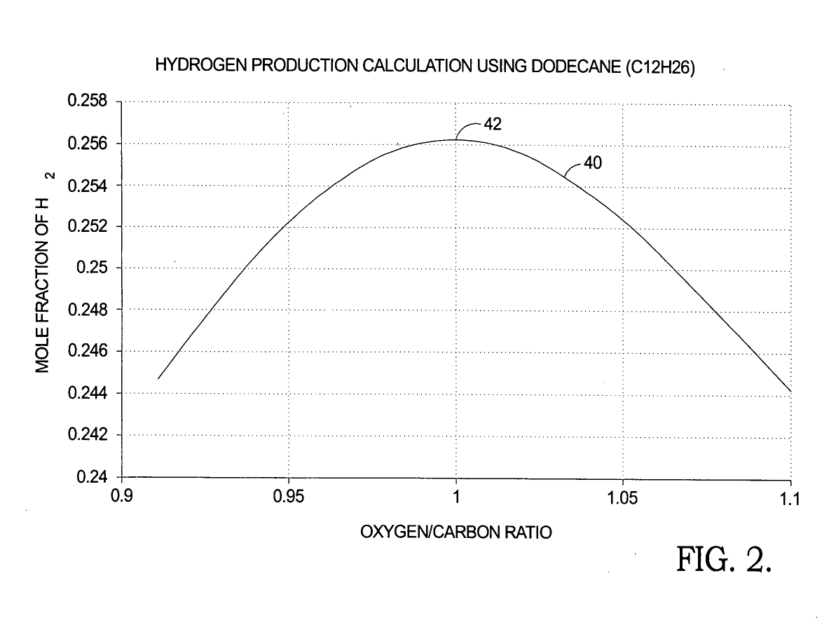

Since the composition of air is known and fixed, and since the carbon percentage of a given hydrocarbon fuel is known, the flow rates of air and fuel define an O/C ratio. Referring to FIG. 2, as an example, curve 40 shows the predicted production of H₂ by a reformer using dodecane (C₁₂H₂₆) as the fuel, with varying O/C ratios. The reformer operating temperature is assumed to be 800° C. The characteristic of peak hydrogen production 42 occurring at a defined O/C ratio is the basis of control system 10. Using a sensor that is responsive to the hydrogen concentration in reformate allows the process to determine where the maximum hydrogen production is occurring. Once the fueling rate has been determined at what fueling rate the maximum hydrogen production has occurred, the fuel rate can be calculated to achieve other O/C ratios if desired.

FIG. 2 shows that, by way of example, reformer 18 produces the maximum concentrations of hydrogen and carbon monoxide when the O/C ratio is 1.0; that is, all carbon is present as carbon monoxide. At O/C ratios less than 1.0, some elemental carbon (soot) and/or hydrocarbon is present; at O/C ratios greater than one, some carbon dioxide is present. The O/C ratio is controlled by the fueling rate and amount of air that is fed into the reformer.

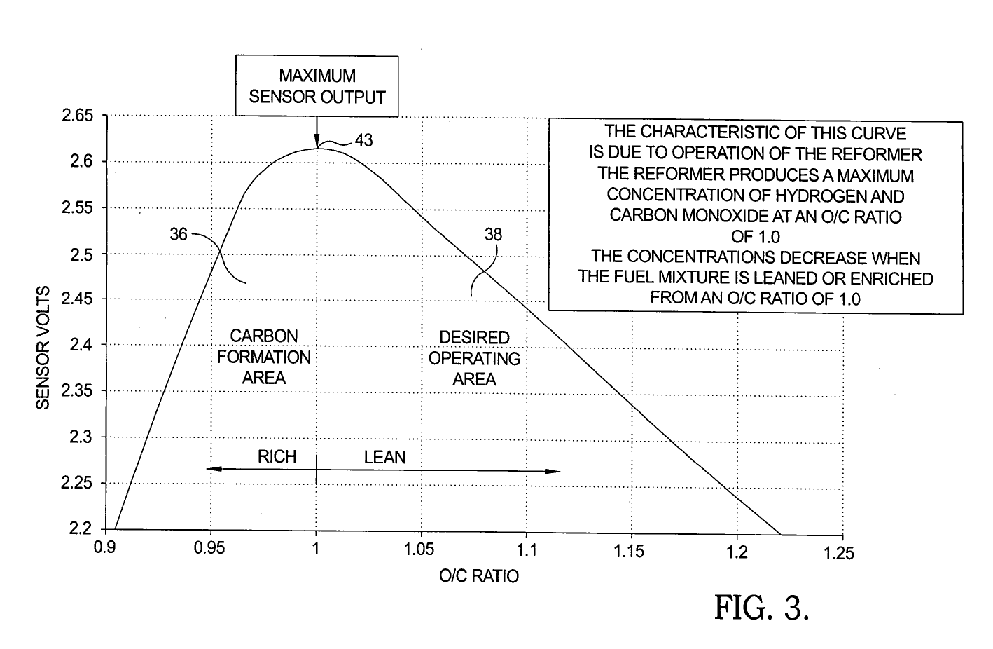

FIG. 3 shows a typical output of a currently-preferred hydrogen sensor. Such sensors are commercially available and also may be readily fabricated from information well known in the prior art. The output signal 34 of sensor 32 is a current source that is converted conventionally into a voltage that can be read by controller 12. To avoid operating reformer 18 in a soot-producing range 36 at O/C<1.0, steady state control is preferably in a desired operating range 38 wherein O/C ratio is biased toward a slight oxygen excess, preferably in a range between about 1.05 and about 1.10.

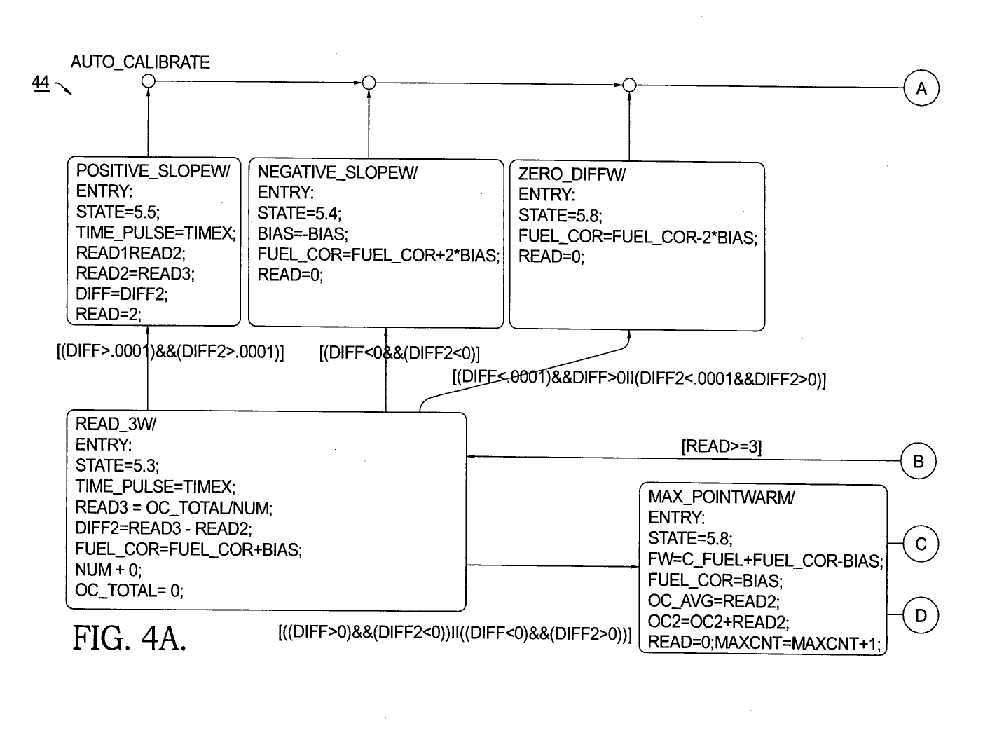

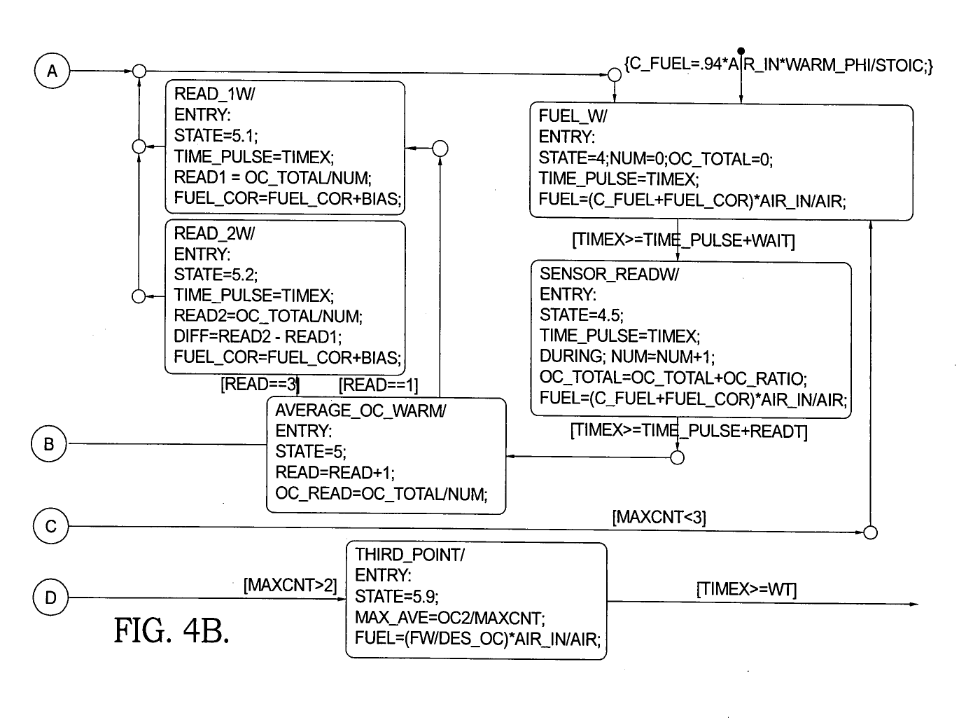

The process of finding the maximum sensor output that occurs at an O/C ratio of 1.0 is performed by varying the fuel rate at a given air flow rate. Readings of the O/C sensor are taken at each fuel rate and used to determine where the maximum sensor reading occurs. FIG. 4 shows a flowchart for an algorithm 44 that finds the maximum sensor reading 42 at a given airflow and fuel rate. Control algorithm 44 is referred to herein as “Auto-Calibrate”.

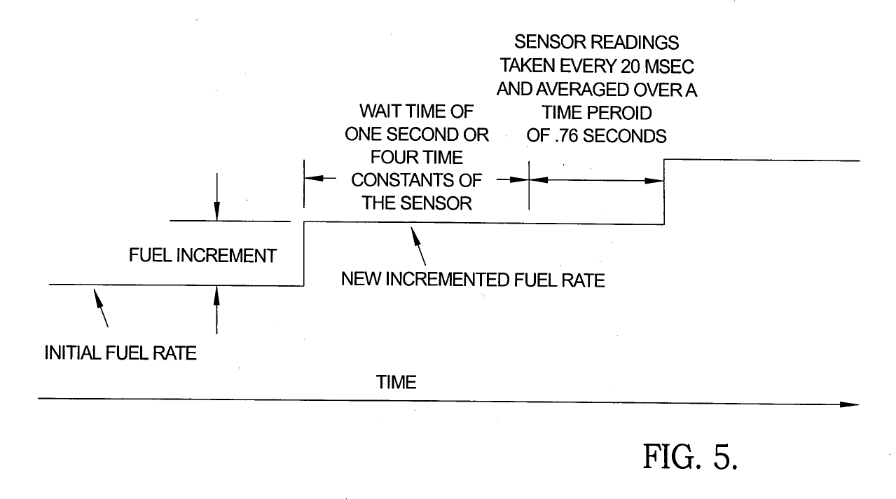

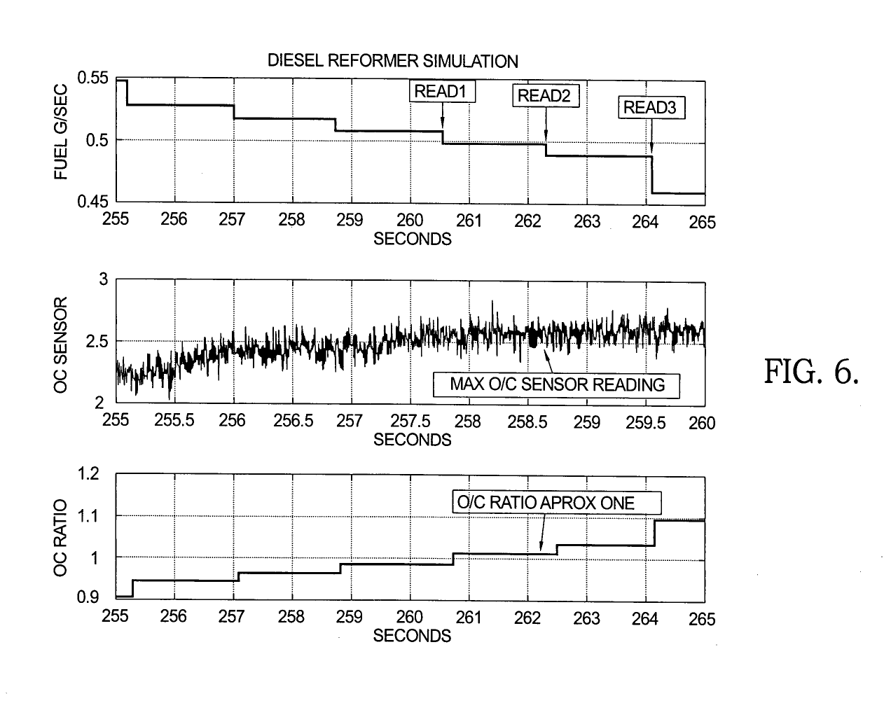

Referring to FIGS. 4, 5, and 6, to find maximum sensor reading 43, auto-calibrate algorithm 44 takes three O/C sensor readings at three different fuel rates. At each sensor reading, the fuel rate is held constant during a waiting period and a reading period. The waiting period allows the reaction to occur in the reformer and the resultant gases to flow downstream to the sensor. The waiting period also allows time for the sensor to measure the content of the resultant gases. A currently-preferred waiting period is one second, which is equivalent to four time constants of the preferred H₂ sensor. The reading period allows for several sensor readings to be taken, accumulated, and averaged. The averaging of several sensor readings reduces the effect of any noise in the system. The values produced for three consecutively incremented fuel rates are shown as read1, read2, and read3. Thus, each reading value is the average of 38 sensor readings over the 0.76 seconds period. Obviously, the waiting period and reading period can be adjusted as desired to match the performance characteristics of the sensor performing the measurements, and the flow rates as necessary. As shown in FIG. 6, the fuel rate is incremented down during this time period. The labels indicate where three consecutive readings occur, and the output is shown for the hydrogen sensor. The corresponding O/C ratios are calculated from the H₂/O/C model programmed into controller 12.

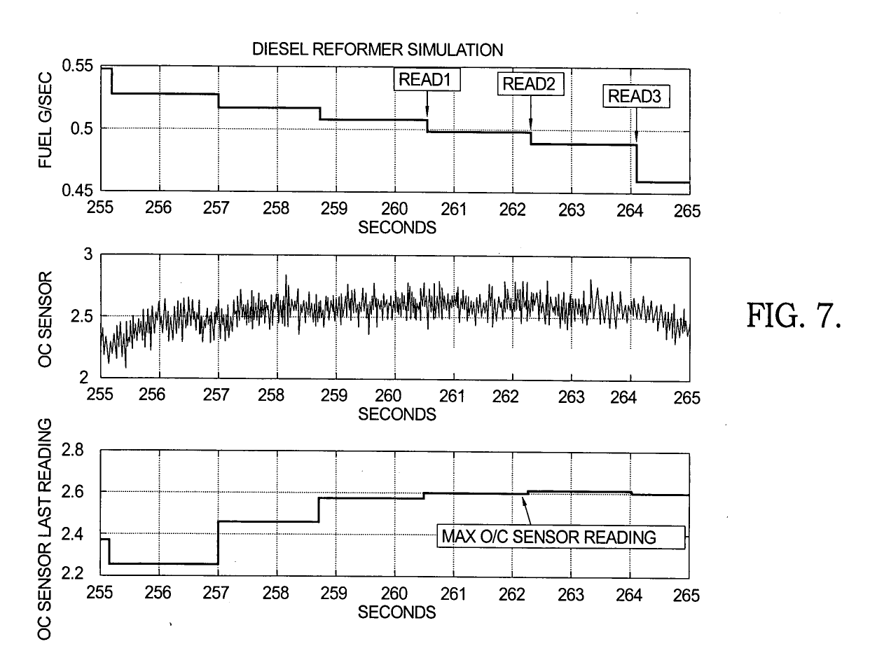

FIG. 7 shows data for the same run but includes the last O/C sensor reading taken by the algorithm in each read period. Reading number 2, which is represented by the label “Read2”, is when the maximum O/C sensor reading takes place. During reformer warm-up mode, the auto-calibrate routine is performed three times. Then an average is taken of the three maximum sensor readings as the value that represents an average sensor value where the O/C ratio is 1.0.

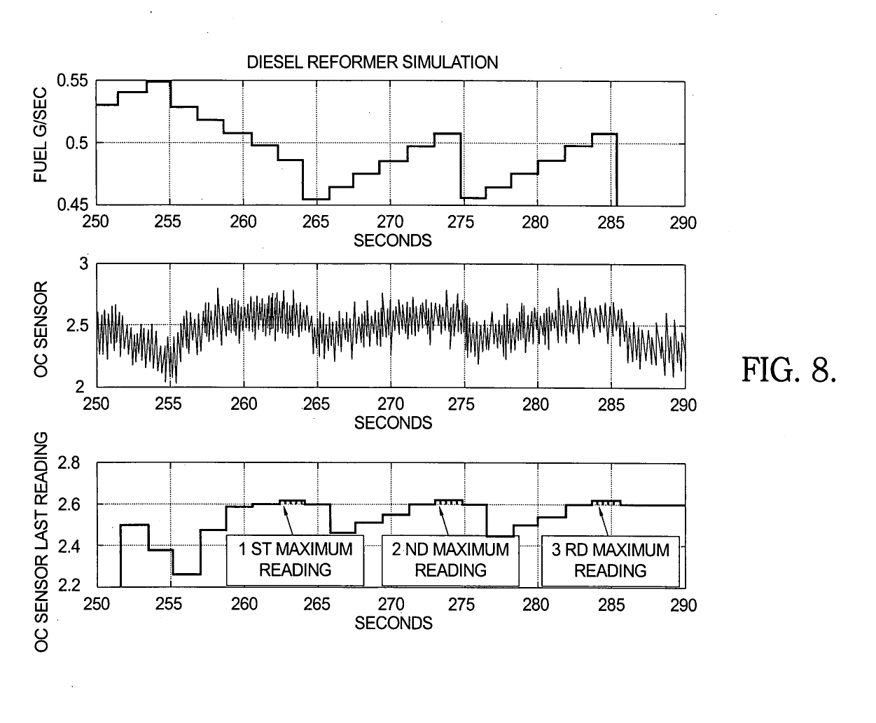

FIG. 8 shows data for a run that includes the three passes during the auto-calibrate routine during reformer warm-up period. It takes approximately 35 seconds to perform the three passes during the auto-calibrate routine. The reformate gas temperature (T6) and the mixing chamber temperature (T3) have to exceed their respective limits before the auto-calibrate mode is entered during the warm-up period. The reformate temperature rises soon after the combustion mode is finished. The mixing chamber temperature takes more time to reach an appropriate value. For this run, the temperature for the auto-calibrate activation mode was set at 750° C. for the reformate gases and 150° C. for the mixing chamber temperature. The auto-calibrate routine could average two readings instead of three if the difference between the first and second maximum readings is very small in magnitude. This would shorten the time for the auto-calibrate mode to about 25 seconds. The time to finish the auto-calibrate mode is also dependent upon how close the initial fueling rate is to the fuel rate to achieve an O/C ratio of 1.0.

The incremental fuel change for seeking the maximum O/C sensor reading during the auto-calibrate mode is set proportional to the airflow rate. In the present case, the airflow rate is multiplied by 0.004 to arrive at the incremental fuel change, which is slightly more than 2% of the total fueling rate for reforming.

A range of incremental fuel rates were examined for the auto calibrate mode. When very small incremental fuel rates were used for the auto-calibrate mode, some erroneous readings were encountered from the O/C sensor. These runs were performed with 15% of noise added to the sensor signal. Incremental fuel rates of 0.5% or less encountered erroneous readings. Using an incremental fuel rate of 2% provides large enough steps to detect the maximum sensor reading and also provide sufficient resolution of the O/C ratio. The 2% incremental rate yields approximately a resolution of ±0.02 H₂ reading around the maximum, corresponding to an O/C ratio range of 0.98 to 1.02 for the fuel control for the sensor curve used. This slope of the O/C sensor curve as it approaches the maximum value determines the resolution.

The above calibration is suitable for a system 10 wherein reformer 18 is operated in a continuous duty cycle. However, for reasons of fuel efficiency, it may be preferable in some applications to generate reformate only periodically (pulsed duty cycle), as required to regenerate NOx trap 26; for example, for five seconds every 30 seconds.

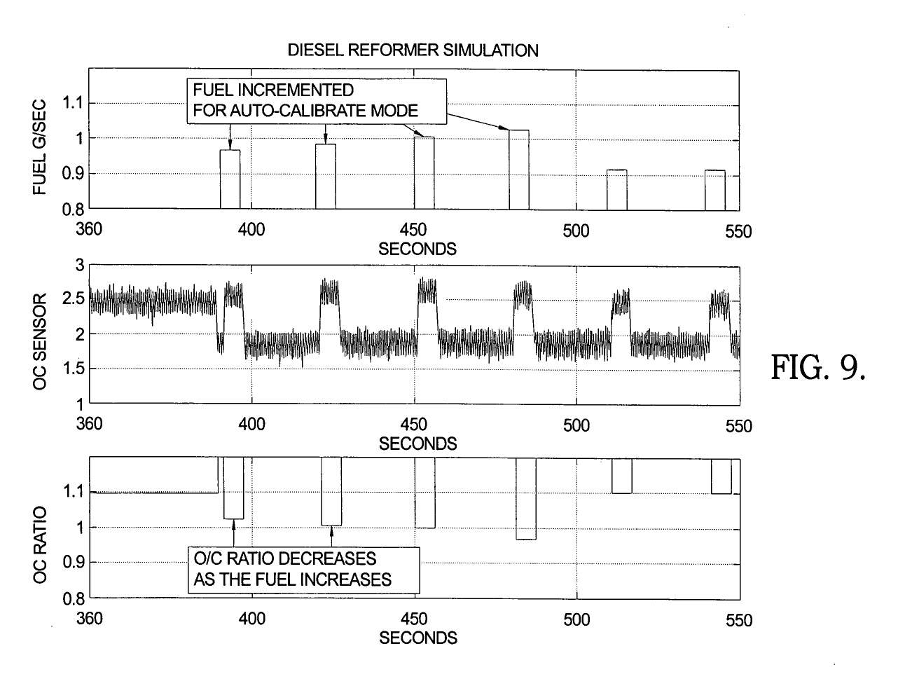

Referring to FIG. 9, auto-calibrate mode is shown for pulsed mode calibration, which takes one O/C sensor reading per pulse. The pulse must be long enough in duration and at the appropriate flow rate to qualify for a sensor reading for the auto-calibration mode. The parameter for the minimum pulse duration for the simulation was set at 5.76 seconds, including a waiting time of five seconds and a sensor reading time of 0.76 seconds. The air flow rate was set to 5.0 g/sec. The reformate temperature (T6) also needs to be in excess of 700° C. after five seconds. If these conditions are met, then the reading is used for the pulsed auto-calibrate mode.

FIG. 9 shows four consecutive pulses at the desired airflow rate to qualify for the auto-calibration mode. The fueling rate is incremented for each pulse and one average reading of the O/C sensor is taken for each pulse.

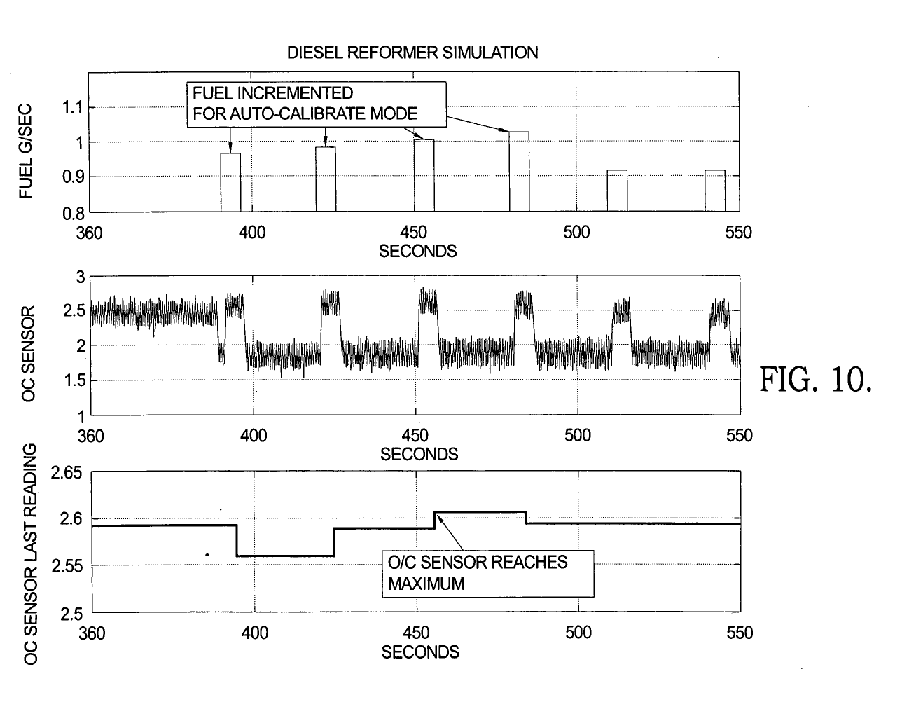

FIG. 10 shows the average O/C sensor readings obtained during the auto-calibrate mode operating in the pulsed operation. The maximum sensor reading occurred at the pulse starting at 450 seconds. This fuel rate is then stored in the variable F1 and is used to calculate the fuel rate for other pulses during the pulsed operation.

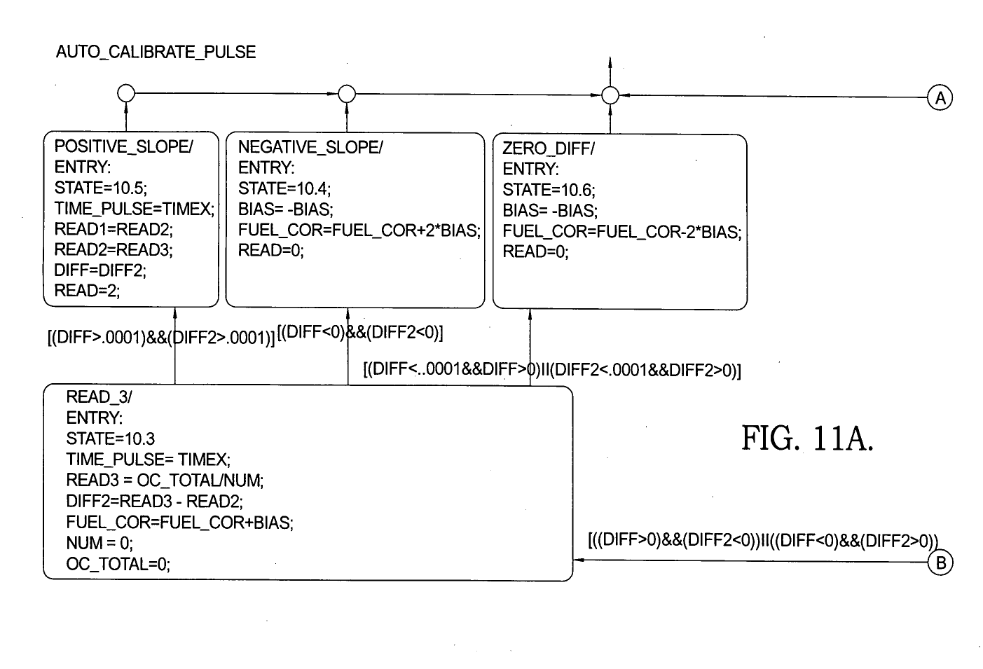

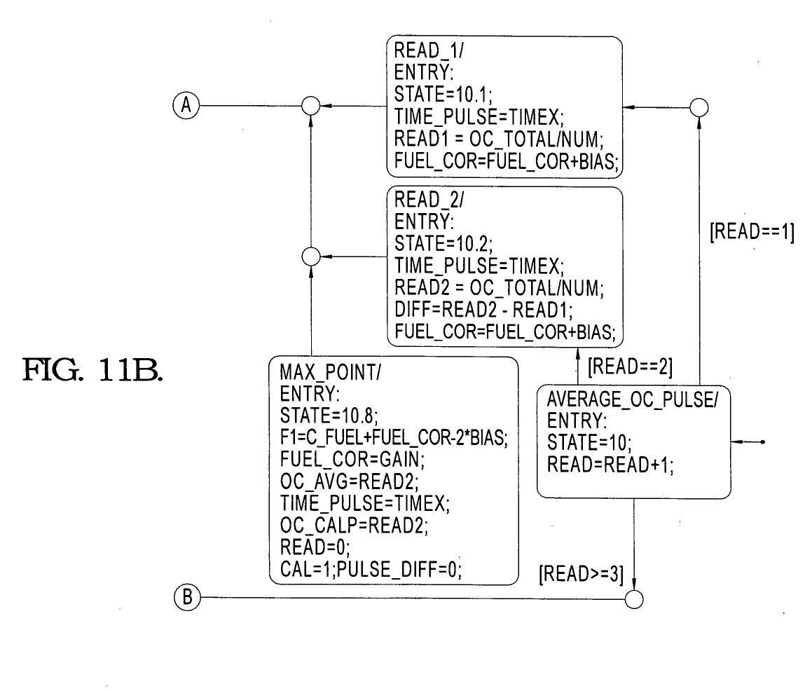

The routine for auto-calibration during pulse mode is very similar to the routine for warm-up auto-calibration. During pulsed operation, there is only one pass made at the maximum O/C sensor reading. FIG. 11 shows the auto-calibration algorithm 50 for the pulsed operation. This routine is performed when the pulse meets the qualifications for the pulse calibration mode. The pulse auto-calibration algorithm uses the same peak detection method as used in the warm-up auto-calibrate algorithm. A separate auto-calibrate mode for pulsed operation is desirable since the pulsed operation is generally performed with higher flow rates than is used for the warm-up mode. The higher flow rates tend to have slightly lower hydrogen concentrations in the reformate gases which will lower the O/C sensor readings compared to the warm-up readings. Performing the auto-calibrate mode during the pulse operation enables the fuel rate to be determined closer to the operating conditions for the pulsed operation.

It has been found that higher airflows produce stabilized output concentrations during pulsed operation. It is suggested to use a calibration airflow value for the pulsed auto-calibrate mode such that the reformer output gases stabilize in six seconds. This helps ensure that the O/C sensor readings that occur at the end of the pulse are valid readings. The pulse duration of six seconds is common during Federal Test Procedures for emissions and fuel economy. Pulsed operation normally occurs during deceleration periods of the test cycles.

After the auto-calibrate mode has determined the fueling rate for the maximum O/C sensor, the reading is used to determine the fuel rate for the following pulses during pulsed operation. The formula used is described as:

Pulse fuel=F1*(Pulse air-flow)/((Auto-Calibrate air-flow)*(Desired O/C ratio))

### Wherein:

F1=fuel rate that produced the maximum O/C sensor reading during the pulsed Auto-Calibrate mode.

Pulse air-flow=measured airflow for pulse operation.

Auto-Calibrate air-flow=measured airflow during Auto-Calibrate pulse mode. Desired O/C ratio=desired O/C ratio for pulsed operation.

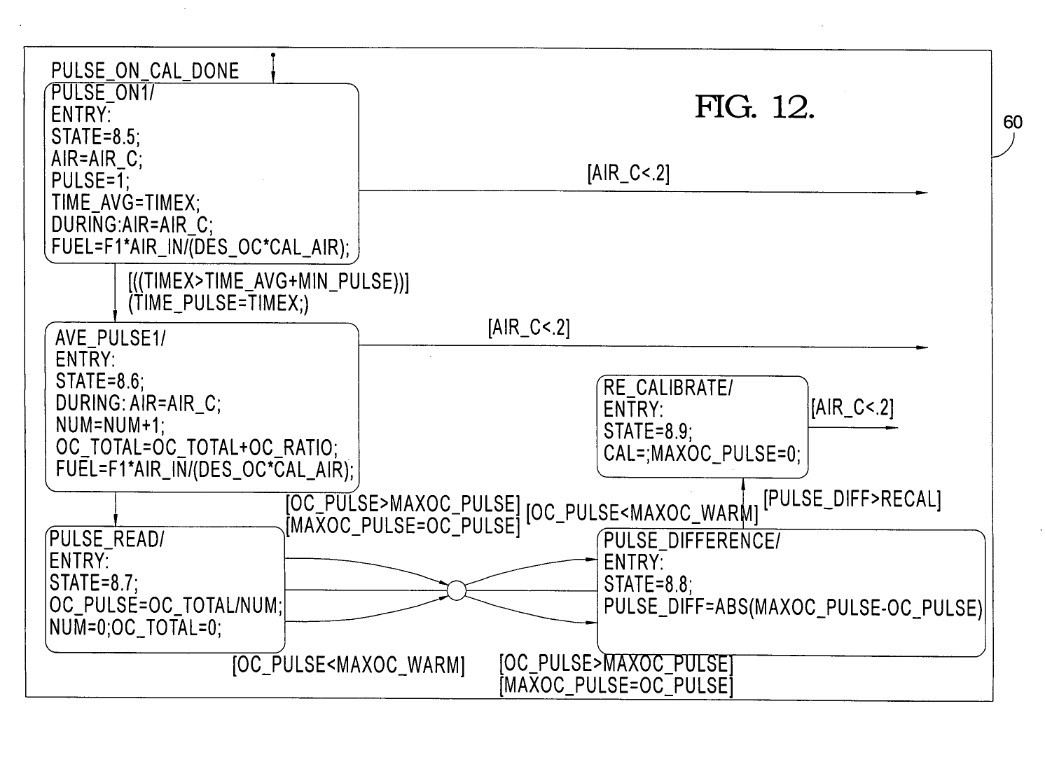

FIG. 12 shows an algorithm 60 for providing fueling rate for pulse mode operation after the Pulse Auto-Calibrate mode has been performed. Algorithm 60 is referred to herein as “Pulse_On_Cal_Done”. This algorithm also tracks the maximum O/C sensor reading during the pulsed operation. It calculates the difference between the maximum reading and the present reading. If the difference is large enough, a re-calibration for the pulse operation is requested. This request causes the auto-calibration for the pulse mode to be run again.

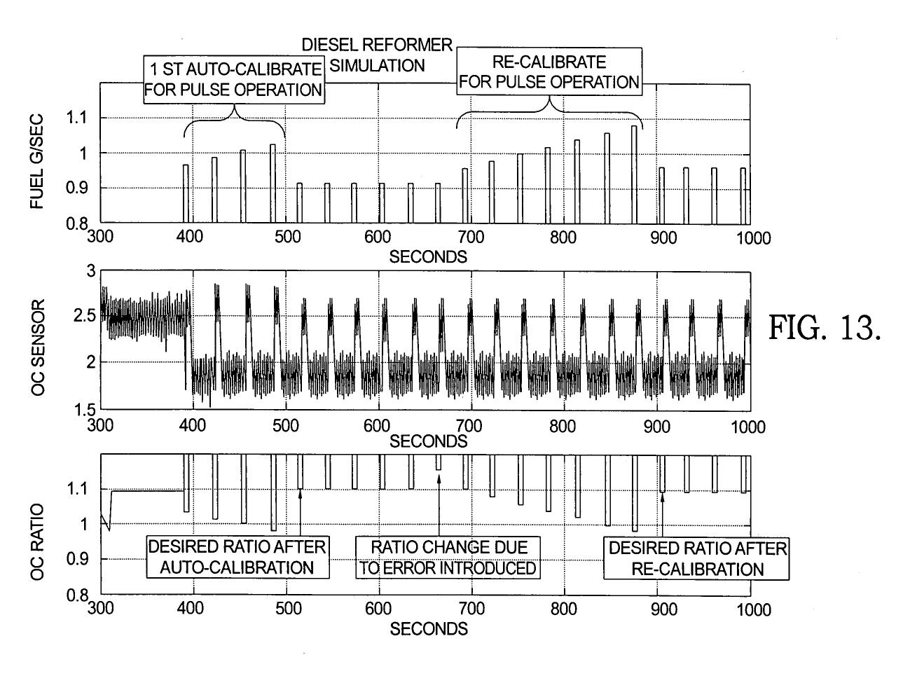

FIG. 13 shows data for a simulated demonstration of re-calibration during pulse mode. The first auto-calibration is started at the 390 second time period. After the calibration is completed, the fuel rate is compensated to produce the desired O/C ratio for the following pulses. The pulses from 510 seconds to 640 seconds produce the desired O/C ratio. Starting at 650 seconds, an artificial error is introduced into the airflow reading, which causes a shift in the O/C ratio of the following pulse. This difference causes the re-calibration to start at the 680 seconds time period. The re-calibration occurs from 680 seconds to 870 seconds. The new fueling rate is established for the following pulses and the O/C ratio returns to the desired ratio after 900 seconds.

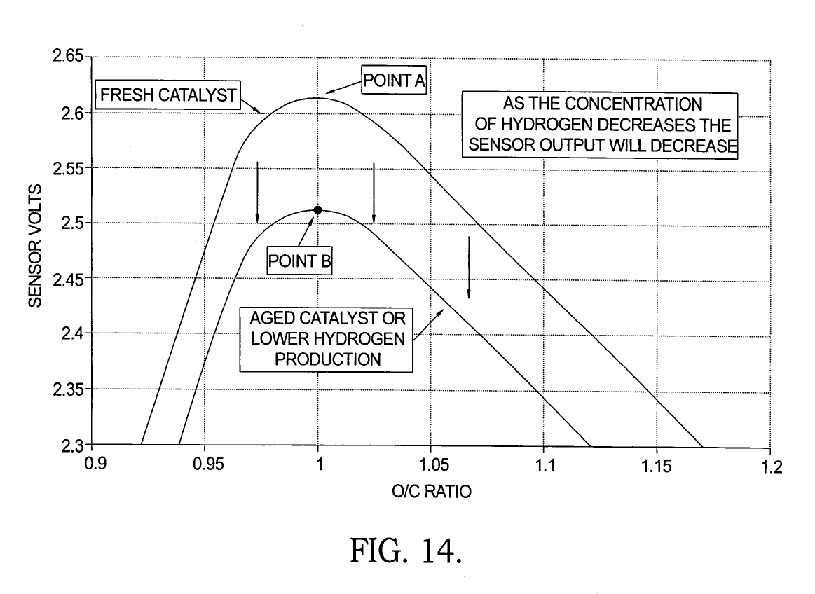

The Auto-Calibrate mode for warm-up and the Auto-Calibrate mode for pulsed operation store the highest O/C sensor reading from the calibrate mode. These values can be used for comparing to later Auto-Calibrate values. If the production of Hydrogen or Carbon Monoxide decreases over time or use, then the O/C sensor readings determined during the auto-calibrate mode will also decrease. FIG. 14 shows the O/C sensor curve movement as the catalyst ages with lower hydrogen production. Point A represents the highest O/C sensor reading when the catalyst is fresh. Point B represents the highest sensor O/C sensor reading when the catalyst has aged. The difference of the sensor values of Point A and Point B is related to the decrease in hydrogen concentration from the output gases of the catalyst. The value of the difference of Point A and Point B can determine if the output needs to be increased by increasing the flow of fuel to increase the amount of hydrogen produced. The difference value can also be used to determine when the catalyst needs to be refurbished or replaced.

Various additives that are used in the diesel fuel industry can affect the O/C ratio at which the maximum hydrogen and carbon monoxide production occurs. Sulfur and aromatics content in diesel fuel have an affect on the production of hydrogen from the reformer catalyst. For example, addition of 100 ppm of dibenzothiophene to the fuel can cause the reformer to produce the maximum concentration of hydrogen at an O/C ratio of 1.16 as compared to 1.0 for pure diesel fuel. Other additives such as toluene, naphthalene, and quinoline also tend to reduce the amount of hydrogen and carbon monoxide formation.

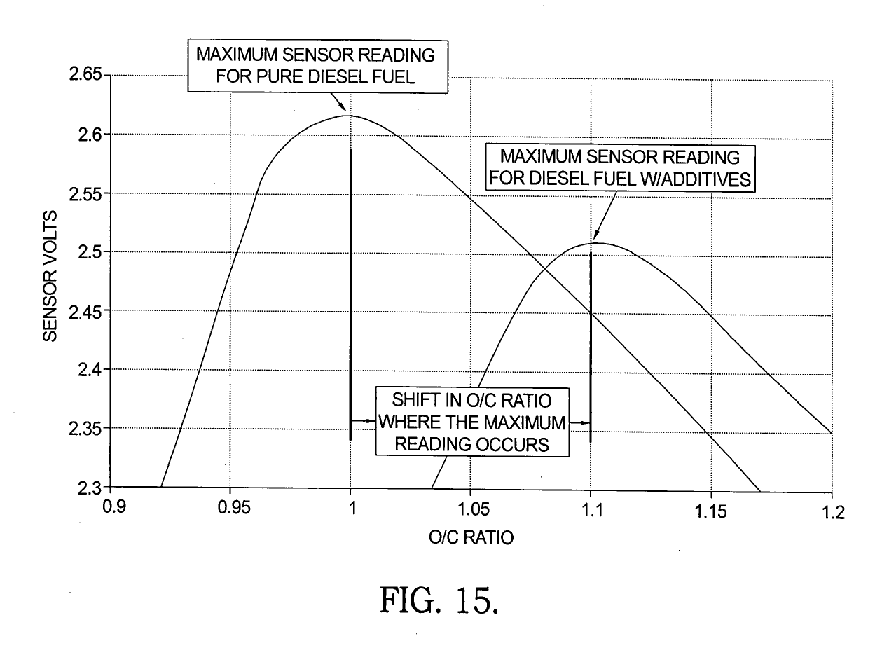

The auto-calibrate mode will still perform properly with additives to the fuel stock. The maximum production of hydrogen may shift with these additives, but the auto-calibrate mode will detect this change. The operating point is able to compensate for the changes in fuel composition. If a shift in the fueling rate corresponds to a lower maximum O/C sensor reading, this might indicate a change in fuel additives. FIG. 15 shows an exemplary shift in maximum O/C ratio with fuel additives.

While the invention has been described by reference to various specific embodiments, it should be understood that numerous changes may be made within the spirit and scope of the inventive concepts described. Accordingly, it is intended that the invention not be limited to the described embodiments, but will have full scope defined by the language of the following claims.

What is claimed is:

1. A system for closed-loop control of oxygen/carbon ratio in reformate being formed from hydrocarbon fuel and air in a catalytic hydrocarbon reformer, comprising:
a) a controllable fuel supply system connected to said reformer;
b) a controllable air supply system connected to said reformer;
c) a hydrogen sensor disposed downstream of said fuel supply system and said air supply system; and
d) a controller connected to said fuel supply system, to said air supply system, and to said hydrogen sensor for receiving input from said hydrogen sensor and responsively setting flow values for fuel and air to provide a predetermined oxygen/carbon ratio in said reformate.

2. A system in accordance with claim 1 wherein said hydrogen sensor is disposed downstream of said hydrocarbon reformer.

3. A system in accordance with claim 1 wherein said predetermined oxygen/carbon ratio is between about 1.05 and about 1.10.

4. A method for setting oxygen/carbon ratio in an air/fuel mixture being supplied to a catalytic hydrocarbon reformer, comprising the steps of:
a) providing a controllable fuel supply system and a controllable air supply system connected to said catalytic hydrocarbon reformer;
b) providing a hydrogen sensor disposed downstream of said controllable fuel supply system and said controllable air supply system;
c) providing a controller connected to said fuel supply system, to said air supply system, and to said hydrogen sensor;
d) setting an air flow rate and a fuel flow rate to form a first air/fuel mixture having a first oxygen/carbon ratio;
e) sending a signal from said hydrogen sensor indicative of said first oxygen/carbon ratio;
f) varying said fuel flow rate to vary said oxygen/carbon ratio;
g) determining a fuel flow rate corresponding to an oxygen/carbon ratio of 1.0;
h) calculating a fuel flow rate productive of a predetermined desired oxygen/carbon ratio; and
i) setting said fuel flow at said calculated fuel flow rate.

5. A method in accordance with claim 4 wherein said predetermined desired oxygen/carbon ratio is between about 1.05 and about 1.10.

6. A method in accordance with claim 4 wherein said determining step is included in an automatic calibration protocol.

7. A method for regeneration of a nitrogen oxides trap in the exhaust stream of a diesel engine, comprising the steps of:
a) providing a catalytic hydrocarbon reformer system for generating reformate containing hydrogen and carbon monoxide, said reformer system including a controllable fuel supply system and a controllable air supply system connected to a catalytic hydrocarbon reformer, a hydrogen sensor disposed downstream of said controllable fuel supply system and said controllable air supply system, and a controller connected to said fuel supply system, to said air supply system, and to said hydrogen sensor;
b) connecting said catalytic hydrocarbon reformer system to said diesel engine such that said reformate may be added to said exhaust stream ahead of said nitrogen oxides trap;
c) setting an air flow rate and a fuel flow rate to form a first air/fuel mixture passing through said reformer and having a first oxygen/carbon ratio;
d) sending a signal from said hydrogen sensor to said control means indicative of said first oxygen/carbon ratio;
e) varying said fuel flow rate to vary said oxygen/carbon ratio;
f) determining a fuel flow rate corresponding to an oxygen/carbon ratio of 1.0;
g) calculating a fuel flow rate productive of a predetermined desired oxygen/carbon ratio;
h) setting said fuel flow at said calculated fuel flow rate to generate reformate having said predetermined desired oxygen/carbon ratio; and
i) entering said reformate having said predetermined desired oxygen/carbon ratio into said diesel exhaust stream ahead of said nitrogen oxides trap according to a predetermined schedule.

8. A method in accordance with claim 7 wherein said predetermined desired oxygen/carbon ratio is between about 1.05 and about 1.10.

9. A method in accordance with claim 7 wherein said predetermined schedule is selected from the group consisting of continuous and pulsed.

10. A method in accordance with claim 9 wherein said pulsed schedule is set for optimal regeneration of said nitrogen oxides trap.

11. A method in accordance with claim 9 wherein said pulsed schedule comprises about five seconds of reformer operation in every thirty seconds.
